(19) United States
(12) Patent Application Publication
(10) Pub. No.: US 2009/0214710 A1
(43) Pub. Date: Aug. 27, 2009

(54) Methods and Compositions For Increasing Fermentation of a Microorganism

(75) Inventors: Dusan Miljkovic, San Diego, CA (US); Jovan Hranisavljevic, Belgrade (YU); Martin Fessenmaier, Aliso Viejo, CA (US)

Correspondence Address: FISH & ASSOCIATES, PC;ROBERT D. FISH, 2603 Main Street, Suite 1000, Irvine, CA, 92614-6232, US

(73) Assignee: VDF FUTURECEUTICALS, INC., Momence, IL (US)

(21) Appl. No.: 12/437,463

(22) Filed: May 7, 2009

Related U.S. Application Data
(62) Division of application No. 10/668,921, filed on Sep. 22, 2003.
Application No. 10/668,921 is a continuation-in-part of application No. 09/802,349, filed on Mar. 8, 2001, now abandoned.
(60) Provisional application No. 60/187,626, filed on Mar. 8, 2000.

(51) Int. Cl. A23L 1/28; C12P 7/06; C12P 7/24; C12P 19/00

(52) U.S. Cl. 426/61; 435/161; 435/147; 435/72

(57) ABSTRACT

Fermentation of a microorganism is increased by including a cytokinin into the fermentation medium. Contemplated cytokinins may be synthetic cytokinins, cytokinins isolated from a natural source, or present in an extract that is specifically enriched for cytokinins. Especially contemplated microorganisms include yeast, and the fermentation in preferred aspects comprises catabolic conversion of a carbohydrate to ethanol and/or carbon dioxide.

This application is a divisional application of U.S. application with the Ser. No. 10/668,921, which was filed Sep. 22, 2003 which is a continuation-in part of U.S. patent application with the Ser. No. 09/802,349 (now abandoned), which was filed Mar. 8, 2001 which claims priority to U.S. provisional patent application with the Ser. No. 60/187,626 filed Mar. 8, 2000. All of which are incorporated by reference herein in their entirety.

### FIELD OF THE INVENTION

The field of the invention is fermentation of microorganisms.

### BACKGROUND OF THE INVENTION

Fermentation is one of the oldest biotechnological processes in which a naturally occurring or synthetic raw material is converted towards a more desirable product. Most fermentation processes employ microorganisms, or extracts and components thereof. For example, malt sugars are converted into alcohol by brewer's yeast in the process of beer brewing. Other fermentation processes utilize yeast cells or yeast cell lysates to generate carbon dioxide for raising dough during bread baking. Still further fermentation processes employ microbial enzymes to convert milk products into yogurt or cheese products.

In order to increase the amount of a desired fermentation product, and/or to decrease the time required for the fermentation to reach a predetermined endpoint, various methods are known in the art. In one common method, the amount of fermenting cells or fermenting enzyme(s) may be increased in the fermentation medium to increase fermentation output or to decrease fermentation time. Although technically relatively simple, increasing the amount of a biocatalyst is often not practicable because the high amount of the biocatalyst tends to interfere with the quality of the desirable product.

In another method, physicochemical parameters may be changed to improve the amount of desired fermentation product. For example, U.S. Pat. No. 4,95,505 to Cho describes fermentation of a microorganism under elevated pressure conditions, and temperature control in a particular temperature range. Alternatively, fermentation conditions may be altered to include a higher oxygen level by increasing oxygen feeding, stir rate, etc. While such methods are conceptually relatively simple and often increase the yield of desirable products, stringent process control typically demands relatively expensive equipment and maintenance.

In yet another method, additives may be included to the fermentation process to stimulate the fermentation of a microorganism. For example, in U.S. Pat. No. 5,362,639 to Griffith et al., the inventors disclose methods to increase anaerobic fermentation rates by addition of condensed phosphates. Although the production of alcohol from sugar increases in such methods, macronutrient elements have to be added in an excessive amount to compensate for the elements sequestered by the condensed phosphates, thereby raising cost and potentially compromising quality of the fermentation product. In yet another example, U.S. Pat. No. 5,486,367 to Fung, an oxygen reactive enzyme is added to a fermentation process to accelerate the fermentation of comestible products. Fung's system allows a relatively wide flexibility in various applications, however, requires relatively expensive enzyme preparations.

Although various methods are known in the art to increase fermentation of a microorganism, all or almost all of them suffer from one or more disadvantages. Therefore, there is still a need to provide compositions and methods to increase fermentation of a microorganism.

### SUMMARY OF THE INVENTION

The present invention is directed to compositions and methods of increasing fermentation of a microorganism. More particularly, especially preferred fermentation media will include a cytokinin-containing preparation that comprises a cytokinin in an amount effective to increase fermentation of the microorganism. Therefore, in especially contemplated methods of increasing fermentation of a microorganism, a cytokinin-containing preparation and a fermentation medium are combined such that the cytokinin-containing preparation (and with that the cytokinin) is present the fermentation medium in an amount effective to increase fermentation of a microorganism.

In one aspect of the inventive subject matter, suitable cytokinin-containing preparations comprise synthetic cytokinins with a purine heterocyclic base (e.g., N⁶-substituted adenine or an optionally N⁶-substituted guanine) or a pyrimidine heterocyclic base (e.g., N⁴-substituted cytosine), preferably at a concentration of greater than 1 microM. Alternatively, or additionally, contemplated cytokinin-containing preparations may comprise a plant extract (e.g., from a plant of the genus Hordeum) and/or yeast extract (e.g., from a yeast of the genus Saccharomyces).

In another aspect of the inventive subject matter, contemplated cytokinins are present in the fermentation medium at a concentration effective to activate an AMP-activated protein kinase of the microorganism (e.g., the SNF-1 protein of yeast) and/or at a concentration effective to increase uptake of a carbohydrate into the microorganism (e.g., via increased expression of a GLUT transporter). Numerous microorganisms are contemplated suitable for use herein. However, particularly preferred microorganisms include those used in the production of ethanol and/or carbon dioxide (e.g., for ethanol fuel, ethanolic beverages for human consumption, or raising dough). Therefore, suitable microorganisms especially include Saccharomyces spec., E. coli, and Zymomonas spec., which may be further genetically modified to enhance production of the desired metabolite (e.g., ethanol, carbon dioxide, etc.). Consequently, suitable fermentation media will include liquid and solid media.

Various objects, features, aspects and advantages of the present invention will become more apparent from the following detailed description of preferred embodiments of the invention.

### DETAILED DESCRIPTION

The inventors have recently discovered that certain barley preparations significantly increase the rate of fermentation of a yeast, and upon further investigation unexpectedly found that at least a portion of the increase in fermentation is attributable to cytokinins present in such extracts. Additional experiments with numerous isolated cytokinins confirmed the inventor's initial discovery.

As used herein the term “fermentation” refers to a process in which one or more substrates present in a fermentation medium are converted by a microorganism (or extract thereof) to a product. Conversion of the substrate may take place in the fermentation medium, however, in more typical aspects of the inventive subject matter, the substrate is imported into and converted within the microorganism to the product, which may then be exported from the microorganism back into the fermentation medium. Contemplated fermentations may be performed utilizing living cells, dormant cells (e.g., freeze-dried cells), or cell extracts. Therefore, the term “increase fermentation” refers to an increase of a desired product in a fermentation (as compared to a fermentation without a cytokinin-containing preparation), wherein at least a portion of the increase in fermentation is attributable to a cytokinin in the fermentation medium.

Consequently, the term “fermentation medium” refers to a medium in which both the substrate as well as the microorganism (or extract thereof) are located such that the microorganism (or extract thereof) can convert the substrate to the desired product. Exemplary fermentation processes include carbohydrate conversion (e.g., various hexoses, or glycerol) to ethanol utilizing a yeast or bacterium in an anaerobic or aerobic manner. In another example, simple and complex carbohydrates are metabolized by a yeast to produce (among other products) carbon dioxide. Of course, a combination of various desirable products are also contemplated (e.g., beer brewing process in which ethanol and carbon dioxide are the products).

As also used herein, the term “cytokinin-containing preparation” refers to any composition of matter that (1) is specifically enriched in at least one cytokinin (e.g., which may naturally occur in that composition), (2) includes an isolated cytokinin (e.g., synthetic cytokinin), and/or (3) that includes a cytokinin isolate (which may comprise numerous non-cytokinin components) prepared from a material that naturally includes a cytokinin. Thus, it should be especially recognized that the term “cytokinin-containing preparation” specifically excludes all materials that naturally comprise cytokinins, provided that such materials are not processed to specifically increase the concentration of a cytokinin (see point (a) above). For example, yeast extract for fermentation of E. coli comprises minor quantities of cytokinins (e.g., as part of tRNA of the yeast). However, as such materials are not prepared to specifically enrich the extract for cytokinins, these materials fall outside the scope of the above given definition for the term “cytokinin-containing preparation”.

As further used herein, the term “cytokinin” refers to a compound that promotes cell division in cells of a plant or in a plant callus, and may further be involved in cell growth, cell differentiation, and in other physiological processes. Especially contemplated cytokinins include those listed below in the section entitled “Contemplated Cytokinins”, and will typically comprise a heterocyclic base, which may be substituted and optionally further be coupled to a carbohydrate radical. Particularly preferred cytokinins will include a purine (e.g., adenine) or pyrimidine (e.g., cytosine) scaffold, and will typically include at least one substituent other than hydrogen on one or more nitrogen atoms in the purine or pyrimidine scaffold. Among other contemplated substituents, particularly preferred substituents include carbohydrate radicals (e.g., various pentose or hexose radicals etc.), alkyl radicals, alkenyl radicals, acyl radicals, acetyl radicals, alkaryl radicals, all of which may include one or more heteroatoms, and/or one or more hydroxyl groups. Another class of contemplated cytokinins includes substituted guanidines and is addressed below.

As still further used herein the term “microorganism” refers to prokaryotic and eukaryotic cells, which grow as single cells, or when growing in association with other cells, do not form organs. Especially contemplated microorganisms include bacteria, yeast, molds, and fungi.

### Contemplated Cytokinin-Containing Preparations

It is generally contemplated that suitable cytokinin-containing preparations include one or more cytokinin, and/or a composition that comprises one or more cytokinin and at least one non-cytokinin compound. Thus, and viewed from another perspective, contemplated cytokinin-containing preparations include all compositions of matter that have been processed to specifically increase the concentration of one or more cytokinins in the composition as well as compositions to which a cytokinin has been added, wherein the cytokinin may be of natural and/or synthetic origin.

There are numerous cytokinins known in the art, and it should be recognized that all known cytokinins are considered suitable for use herein. However, particularly preferred cytokinins will include those according to Formula I or Formula II,

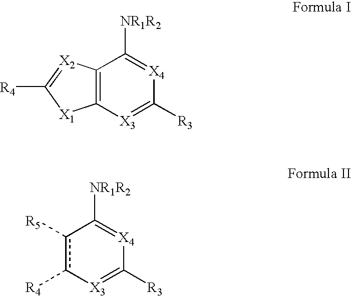

wherein X₁, X₂, X₃, and X₄ are independently NR₆ or CR₆R₇, and wherein at least one of X₁, X₂, X₃, and X₄ is NR₆; wherein R₁ and R₂ are independently hydrogen, alkyl, alkenyl, alkynyl, alkaryl, aryl, all of which may include one or more heteroatoms (e.g., S, N, O, P), and optionally one or more functional groups. Similarly, R₃, R₄, and R₅ are independently halogen, CN, OH, NH₂, H, alkyl, alkenyl, alkynyl, alkaryl, aryl, all of which may include one or more heteroatoms (e.g., S, N, O, P), and optionally one or more functional groups. R₆ is null, a carbohydrate (typically a monosaccharide, and most preferably a pentose or hexose), or R₁; R₇ is R₁.

Thus, the term “functional groups” refers to groups including nucleophilic groups (e.g., —NH₂, —OH, —SH, —NC, —CN etc.), electrophilic groups (e.g., C(O)OR, C(X) OH, C(Halogen)OR, etc.), polar groups (e.g., —OH), non-polar groups (e.g., aryl, alkyl, alkenyl, alkynyl, etc.), ionic groups (e.g., —NH₃⁺), and halogens, as well as NHCOR, NHCONH₂, NHCSNH₂, OCH₂COOH, OCH₂CONH₂, OCH₂CONHR, OC(Me)₂COOH, OC(Me)₂CONH₂, NHCH₂COOH, NHCH₂CONH₂, NHSO₂R, NHSO₂CF₃, OCH₂-heterocycles, PO₃H, SO₃H, (CH₂)₁₋₃COOH, CH═CHCOOH, O(CH₂)₁₋₄COOH, NHCOCH₂CH(OH)COOH, CH(COOH)₂, CH(PO₃H)₂, OCH₂CH₂CH₂COOH, NHCHO etc., wherein R is hydrogen, alkyl, alkenyl, alkynyl, alkaryl, aryl, all of which may include one or more heteroatoms (e.g., S, N, O, P).

Where appropriate, it should also be recognized that contemplated cytokinins may include asymmetric centers and/or conjugated bond systems, and may therefore be present in one or more different configurations and/or conformations. Thus, it should be recognized that all stereoisomers (e.g., diastereomers, enantiomers), tautomers (e.g., keto/enol tautomeric forms), and mixtures thereof are contemplated herein. Similarly, contemplated compounds may form salts with acids or bases, and all such salts are specifically included herein.

Particularly preferred cytokinins include N⁶-benzyladenine, N⁶-benzyladenosine, N⁶-benzyladenine-3-glucoside, N⁶-benzyladenine-7-glucoside, N⁶-benzyladenine-9-glucoside, N⁶-benzyl-9-(2-tetrahydropyranyl)adenine, N⁶-benzyladenosine-5′-monophosphate, N⁶-gamma, gamma-dimethyl-allyl-aminopurine, dihydrozeatin, dihydrozeatin riboside, dihydrozeatin-7-beta-D-glucoside, dihydrozeatin-9-beta-D-glucoside, dihydrozeatin-O-glucoside, dihydrozeatin-O-glucoside riboside, dihydrozeatin riboside-5′-monophosphate, dihydrozeatin-O-acetyl; N⁶-isopentenyladenine, N⁶-isopentenyladenosine, N⁶-isopentenyladenosine-5′-monophosphate, N⁶-isopentenyladenine-7-glucoside, N⁶-isopentenyladenine-9-glucoside, 2-methylthio-N⁶-isopentenyladenosine, 2-methylthio-N⁶-isopentenyladenine, 2-thio-N⁶-isopentenyladenine, 2-benzylthio-N⁶-isopentenyladenine, 2-isopentenylamine, kinetin, kinetin riboside, kinetin-9-glucoside, kinetin riboside-5′-monophosphate, meta-topolin, meta-topolin riboside, meta-topolin-9-glucoside, ortho-topolin, ortho-topolin riboside, ortho-topolin-9-glucoside, trans-zeatin, trans-zeatin riboside, cis-zeatin, cis-zeatin riboside, trans-zeatin-7-glucoside, trans-zeatin-9-glucoside, trans-zeatin-O-glucoside, trans-zeatin-O-glucoside riboside, trans-zeatin riboside-5′-monophosphate, trans-zeatin-O-acetyl, 2-chloro-trans-zeatin, 2-methylthio-trans-zeatin, and 2-methylthio-trans-zeatin riboside.

Further suitable nucleoside-type cytokinins are described by Mok et al in Annu. Rev. Plant Physiol. Plant Mol. Biol. 2001, 52: p 89-118, which is incorporated by reference herein. Moreover, where non-nucleoside-type cytokinins are preferred, cytokinin analogs may be employed, and such compounds are exemplarily described in U.S. Pat. Nos. 4,822,408 and 4,677,226 to Lutz et al., and in U.S. Pat. No. 4,594,092 to Speltz et al, all of which are incorporated by reference herein. Thus, it should be recognized that all compounds with cytokinin activity (e.g., purine-type cytokinins, pyrimidine-type cytokinins, cytokinin analogs, including substituted guanidines and substituted ureas) are deemed suitable for use in conjunction with the teachings presented herein. Cytokinin activity may be identified using the tobacco callus test as described in Physiol. Plant 1965, 18: 100-127, or J. Biol. Chem. 1975, 250 (18): 7343-7351, and especially preferred compounds will lead to calli with an at least 10% increase (at optimum concentration of the added cytokinin or cytokinin analog) in weight of the callus as compared to the control (i.e., without added cytokinin).

It should be recognized that most of the contemplated cytokinins are known compounds that are commercially available, or may be prepared following procedures well known in the art. For example, synthesis of various alkenylpurine cytokinines is described in Bioorg Med. Chem. 2002, 10 (5):1581-6, while N⁶-disubstituted cytokinins may be prepared as discussed in Chem. Pharm. Bull. (Tokyo). 1994; 42 (5): 1045-9. Further synthetic procedures for the preparation of various cytokinins may be found in Nucleic Acids Symp. Ser. 1980; (8):s27-30, and Biochemistry 1973, 5; 12 (12):2179-87. Alternatively, contemplated cytokinins may also be prepared in pure form or as a cytokinin-enriched extract from various sources (e.g., plants, calli, yeasts, bacteria, etc., all of which may be recombinant or native). Typical procedures for isolation and/or enrichment are described in Acta Microbiol Pol. 1980; 29 (2): 117-24 (from bacteria), Acta Microbiol Pol. 1985; 34 (2):177-85 (from fungus), and FEBS Lett. 2003; 533 (1-3): 63-6 (from plants). Of course, it should be recognized that isolated and/or synthetic cytokinins may be employed directly as cytokinin-containing preparation, or may be admixed to a carrier or dissolved in a solvent or solvent mixture to form the cytokinin-containing preparation.

It is especially preferred that where the cytokinin is not synthetically prepared or purchased, a cytokinin-containing preparation is produced from a plant or yeast. For example, particularly suitable plants include those belonging to the genus Hordeum, and most preferably Hordeum vulgare. An exemplary preparation for such an extract is given below in the section entitled “Experiments and Data”. Alternatively, however, it should be recognized that numerous other plants are also suitable for extraction of cytokinins or for production of contemplated cytokinin-containing preparations. Suitable plants will preferably include members of the poaceae family, but also higher plants and parts thereof. Alternatively, where yeast is employed as a starting material for contemplated cytokinin-containing preparations, Saccharomyces spec. is most preferably employed as material for production of contemplated cytokinin-containing preparations.

Still further contemplated cytokinins and cytokinin analogs for use in conjunction with the teachings presented herein are described in our co-pending provisional application with the Ser. No. 60/499,637, filed Sep. 2, 2003 (Title: Cytokinins And Cytokinin Analogs As Therapeutic Agents) and provisional patent application with the Ser. No. 60/493,447, filed Aug. 8, 2003 (Title: Compounds And Compositions That Modulate Metabolism, Their Use And Related Methods), both of which are incorporated by reference herein.

While it is generally preferred that contemplated cytokinins are chemically substantially pure (i.e., concentration of contemplated cytokinins greater than 90 wt %, preferably greater than 95 wt %, most preferably greater than 99 wt %), it should also be appreciated that a cytokinin may also be coupled to one or more non-cytokinin molecule, and particularly contemplated non-cytokinin molecules include thaumatin-like proteins. Thus, contemplated cytokinin-containing preparations especially include complexes between a cytokinin and thaumatin-like proteins.

### Contemplated Fermentations

It is generally contemplated that the cytokinin-containing preparations according to the inventive subject matter can be used in numerous fermentation processes, including those in which the fermentation medium is a liquid (which may contain suspended solids) and those in which the fermentation medium is a non-liquid (which may contain a liquid to at least some degree).

Depending on the particular desired fermentation product and the specific microorganism employed, it should be recognized that the fermentation medium and fermentation procedure will vary substantially. For example, where the desired fermentation product is continually produced by a yeast or bacterium, suitable fermentations include batch-feed, continuous feed, and non-fed fermentations. On the other hand, where microbial production of the desired fermentation product is dependent on the presence of a specific inductor at a particular fermentation stage (e.g., specific cell density), suitable fermentation processes will include batch fermentations. Exemplary guidance for suitable fermentation can be found in “Principles of Fermentation Technology” by Stephen J. Hall, Peter F. Stanbury, and Allan Whitaker (Butterworth-Heinemann; 2nd edition, ISBN: 0750645016), which is incorporated by reference herein.

In one preferred aspect of the inventive subject matter, suitable fermentation products include those with nutritional, pharmaceutical, and/or industrial value. For example, preferred fermentation products include various alcohols (e.g., methanol, ethanol), carboxylic acids (e.g., acetic acid, or lactic acid), amino acids (e.g., tryptophan, histidine), nucleosides and oligo-/polynucleotides (e.g., adenine, tRNA, mRNA, etc.), recombinant and non-recombinant proteins (e.g., recombinant antibodies, native enzymes, etc.). Consequently, the fermentation in numerous embodiments will typically employ an aqueous fermentation medium with a carbohydrate as a substrate that is converted or incorporated into the desired product by the microorganism. Alternative substrates will at least in part depend on the particular nature of the desired product and/or the metabolic pathways in the particular microorganism. Of course, it should be recognized that suitable fermentations may be aerobic, micro-aerobic, or anaerobic.

Suitable fermentation media will therefore include minimal media sustaining growth of a microorganism in which the substrate is a carbohydrate, preferably a monosaccharide (e.g., glucose, fructose, etc.), or glycerol. Alternatively, enriched media may be employed where the particular microorganism will have a specific demand for one or more nutrients. Depending on the particular microorganism, there are numerous fermentation media known in the art, and suitable media can be found for a variety of microorganisms (e.g., fermentation media commercially available from ATCC, P.O. Box 1549, Manassas, Va. 20108). In further particularly preferred aspects, it should be appreciated that the fermentation medium may also include one or more non-synthetic components, and especially preferred fermentation media include those comprising milk or a component thereof, or those comprising malted barley (or extracts/components thereof). For example, where the fermentation is employed to produce a milk product (e.g., yogurt, small curd cheese, soft cheese, hard cheese, etc.), the fermentation medium may predominantly comprise processed or raw milk. On the other hand, where the fermentation is employed to produce an alcoholic beverage, the fermentation medium may predominantly comprise processed or unprocessed fruit juice (e.g., grape, apple, etc.), or other plant material with fermentable carbohydrate content (e.g., malted barley and/or wheat).

Consequently, the choice of the microorganism employed in contemplated fermentations will vary, and will at least in part depend on the desired product. For example, where ethanol production is especially desired, suitable microorganisms include those of the genus Saccharomyces, Escherichia, and Zymomonas (all of which may be recombinant). On the other hand, where nucleoside and nucleoside analog production is especially desired, suitable microorganisms include those of the genus Klebsiella. In a still further example, where antibiotics are produced, suitable microorganisms include those of the genus Streptomyces.

Contemplated cytokinin-containing preparations may be added to the fermentation medium at various times, however, it is generally preferred that the cytokinin-containing preparations are added to the fermentation medium at or preceding inoculation of the fermentor. Alternatively, or additionally, the cytokinin-containing preparations may also be added during the fermentation process (i.e., after inoculation of the fermentation medium). It should be recognized that a person of ordinary skill in the art will readily determine the appropriate point of time of addition of cytokinin-containing preparations.

With respect to the concentration of contemplated cytokinin-containing preparations in the fermentation medium, it is generally preferred that addition of the cytokinin-containing preparation will result in a total concentration of the cytokinin (or cytokinins) in the fermentation medium of at least 1 microM, and most typically between about 5 and about 50 microM (The term “about” as used herein in conjunction with a numeral refers to a range of +/−10%, inclusive, of that numeral). However, in alternative aspects, the concentration of the cytokinin may be less than 1 microM, especially where the cytokinin-containing preparation is added in a continuous fashion. On the other hand, the cytokinin concentration in the fermentation medium may also be significantly higher than 1 microM, and suitable contemplated concentrations include those between about 50 microM and 200 microM (and even higher), where the cytokinin has a relatively low fermentation enhancing activity, or where the cytokinin is readily degraded in the medium.

Where the cytokinin-containing preparation comprises an extract that is enriched in one or more cytokinins (e.g., malted barley extract, or yeast extract), it should be recognized that the appropriate amount of the extract can be determined by a person of ordinary skill in the art without undue experimentation. In general, however, it is contemplated that the suitable amount of such an extract will correspond to an amount in which addition of the extract to the fermentation medium will result in a concentration of a cytokinin (provided by the extract) of at least 1 microM.

Where the fermentation medium is not a fluid, (e.g., dough in bread fermentation, or soil in bioremediation), addition of the cytokinin-containing preparation will follow similar considerations as provided above. Thus, addition of a cytokinin will typically be in the range of between about 0.001 wt % to about 1 wt % of the total weight of the non-liquid fermentation medium.

### Experiments and Data

The following examples provide various experimental procedures to prepare and use compounds and compositions according to the inventive subject matter. Examples 1-3 describe the production of exemplary cytokinin-containing preparations, while examples 4-6 describe the biological activity of the preparations of examples 1-3.

### Exemplary Cytokinin-Containing Preparation

### I

Various stock solutions of commercially available cytokinins were prepared such that the cytokinin was present in the stock solution at a concentration of about 1 mM in suitable solvent (e.g., water, methanol, DMSO). The so prepared stock solutions were then added to the fermentation medium at various concentrations to determine optimum enhancing concentrations.

### Exemplary Cytokinin-Containing Preparation

### II

Barley grains were malted according to procedures well known in the art of beer brewing (see e.g., Principles of Brewing Science, Second Edition, by George J. Fix; Brewers Publications; ISBN: 0937381748, or The Brewers' Handbook by Ted Goldhammer; KVP Publishers; ISBN: 0967521203). In order to extract soluble substances from the malt and to convert additional insoluble solids into soluble material through controlled enzymatic conversion, a step of mashing was subsequently applied to the ground malt (suspended in water) according to a typical brewer's schedule. The temperature cycles were as follows: Incubation at 40° C. for 60 min, incubation at 50° C. for 60 min, incubation at 60° C. for 60 min, incubation at 72° C. for 60 min, and incubation at 75°-80° C. for 60 min. Soluble portions of samples were separated from husks and other insoluble material and freeze-dried.

The freeze-dried barley extract obtained after mashing at 40° C. served as base for fractionation into its components. A first fractionation was achieved by preparative liquid chromatography using a DEAE-Sephacel column (2.6×20 cm) equilibrated with 50 mM phosphate buffer, pH 7.8. 150 mg of the freeze-dried sample was dissolved in 10 ml of buffer and placed on the column. A linear NaCl-gradient (0-0.5 M) was run at a flow rate of 10 ml/h. Fractions (2 ml each) were collected, and elution was monitored at 280 nm. The DEAE chromatography resulted in four distinct protein peak fractions: I—basic, II—neutral, III— and IV—acidic. Respective peak fractions were collected, desalted and concentrated by membrane ultra-filtration using a membrane cut-off pore size of 1000 Dalton, and concentrated corresponding fractions were checked for their capacity to influence yeast fermentation rate. The basic fraction I produced significant inhibitory effect (i.e., a reduction of the yeast fermentation rate), while the remaining three concentrated fractions were almost inert. As it could later be identified (data not shown), the main proteinaceous component in fraction I represent thaumatin-like proteins. It has been noticed during the membrane ultra-filtration of the pooled protein fractions I-IV (i.e., fractions obtained by ion exchange chromatography), that the filtrate of some fractions contains LMW (low molecular weight) substances with a UV absorbance maximum of approximately 260 nm. These observations prompted us to employ molecular sieving chromatography to separate these LMW substances from proteins in these fractions.

For that purpose, the four separated fractions by DEAE-Sephacel column I-IV were pooled and freeze-dried. Molecular sieving chromatography was performed on Sephadex G-75-50 column (2.8×80 cm) with 50 mM phosphate buffer, pH 7.8, containing 0.5 M NaCl (flow rate—12 ml/h, fractions 2 ml, elution recorded at 260 nm). LMW compounds with an absorbance near 260 eluted at relatively high elution volume. Where the separated fractions were individually subjected to molecular sieving on a Sephadex G-75-50 column, LMW compounds eluted near to the end of the separation, typically between 60th-80th fractions. These fractions were designated GMM-1, GMM-2 and GMM-4, and consist of LMW components. All of GMM-1, GMM-2 and GMM-4 increased the rate of yeast fermentation.

### Exemplary Cytokinin-Containing Preparation

### III

20 g of freeze-dried barley extract obtained after mashing at 40° C. was suspended in 80 ml of water and stirred over night at ambient temperature. The suspension was supplemented with 120 ml of 0.8 M NaCl solution and extraction was continued for 24 hours with stirring. An aqueous extract was separated from the suspension by vacuum filtration over a cellulose filter pad.

The filtered extract was freeze-dried or vacuum-evaporated. So obtained dry malt extract (yield approx. 12-14 g) contained 5.6 g of NaCl originating from the extracting solvent and a complex mixture of water soluble barley components. The filtered freeze-dried extract was purified by extraction with two 50 ml portions of warm ethanol under vigorous mixing for two hours. The ethanolic extracts were filtered, combined, and evaporated to an oily residue in vacuum. The oily residue was re-dissolved in 15 ml of water and freeze-dried, resulting in a hard glassy yellowish product in a total amount of approximately 3 g. The glassy yellowish product increased the rate of yeast fermentation.

### Increase in Fermentation Using Selected Cytokinins

### IV

Biological activity of numerous exemplary preparations according to the first example (I) was monitored by quantification of brewers' yeast fermentation rate under anaerobic conditions using a modified Warburg method (Mirsky, N. et al., J. Inorg. Biochem. 13 (1):11-21 (1980), incorporated by reference herein): Two grams of wet brewers yeast cells (about 20% dry weight) were suspended in fermentation medium (25 ml of 60 mM phosphate buffer, pH 5.7 and 10 ml of 5% (w/v) glucose solution), and aliquots of the products from example II or III were added to the fermentation medium for testing. Incubations were carried out in 50 ml fermentation flasks at 25° C. for 60 minutes. The fermentation rates were measured from the volume of generated carbon dioxide. Table 1 below depicts the results for the exemplary cytokinins tested.

| | | FERMENTATION |
|---|---|---|
| | | INCREASE |
| | | (after 60 minutes; |
| CYTOKININ | [c] in microM | control = 1) |
| Cis-Zeatin | 6.2 | 1.44 |
| Trans-Zeatin | 8.9 | 1.17 |
| Dihydro-Zeatin | 9.1 | 1.74 |
| Benzyl-Adenine | 11.3 | 1.74 |
| Gamma,gamma-dimethylallyl-6- | 21.3 | 1.92 |
| aminopurine | | |
| Kinetin Riboside | 101 | 1.67 |
| N⁶-acetyl-Adenosine | 30.7 | 1.92 |
| N²-acetyl-Guanosine | 13.3 | 1.98 |
| N⁴-acetyl-Cytidine | 15.1 | 2.05 |
| AICAR | 135.5 | 1.77 |

### Increase in Fermentation Using Selected Cytokinin Preparations

### V

The biological activity of LMW fractions from exemplary preparation of example (II) (GMM-1, GMM-2 and GMM-4) and the glassy yellowish product from exemplary preparation of example (III) was monitored by quantification of brewers yeast fermentation rate under anaerobic conditions as described above. All of the tested LMW fractions or the product from Examples (II) and (III) showed significant biological activity, and the results are summarized in Table 2 below.

| | | FERMENTATION |
|---|---|---|
| | | INCREASE |
| CYTOKININ | | (after 60 minutes; |
| PREPARATION | [c] in mg/l | control = 1) |
| GMM-1 | 13.25 | 1.80 |
| GMM-2 | 13.25 | 1.17 |
| GMM-4 | 6.1 | 1.06 |
| Total Malted Barley Extract | 25.3 | 1.79 |

In a further experiment, the activity of GMM-2 was tested under aerobic conditions. Despite general restriction of yeast fermentation caused by combined effects of NaCl from buffer and air oxygen (Pasteur effect), the relative amount of generated carbon dioxide was doubled in comparison to the included control (data not shown).

### Contemplated Mechanisms

It is known in the art that glucose deprivation in various eukaryotic cells will induce an Akt-dependent synthesis and incorporation of GLUT-1, and possibly also GLUT-4 into the cell membrane of a glucose deprived cell (see e.g., Eur. J. Cell Biol. (2000), 79: 943-949). As such a mechanism appears to be evolutionarily highly conserved, the inventors contemplate that the increase in fermentation of a microorganism, and especially where a carbohydrate is present in the medium, may also be present in yeast, and possibly even in certain bacteria. Of course, it should be recognized that the yeast and/or bacterium will have the corresponding Akt analog (which will be expected to have a relatively high degree of sequence homology with Akt). Consequently, while not wishing to be bound by any specific theory of hypothesis, the inventors contemplate that at least part of the increase in fermentation is attributable to an increased glucose (or other carbohydrate) uptake into the microorganism, wherein such uptake may be effected by a carbohydrate-specific transporter (e.g., GLUT-1 or GLUT-4 homolog in yeast). There are numerous such carbohydrate-specific transporters known for yeast, and all of such transporters are contemplated herein (see e.g., FEMS Microbiol. Rev. 1997, 21 (1): 85-111).

Based on further in vitro experiments on muscle cells (data not shown), the inventors further contemplate that SNF-1, which is the yeast analog to mammalian AMPK, is activated by cytokinins, and will as a consequence result in specific modulation of the metabolism of the microbial organism (It is generally known that relatively high levels of 5′-AMP relative to ADP/ATP will activate mammalian AMPK, which is believed to be an essential regulatory component in the energy balance of a cell). Therefore, the inventors contemplate that the increase in fermentation by cytokinins may be at least in part attributable to activation of the microbial analog of AMPK (e.g., SNF-1 in yeast). Numerous experiments supporting such theories are described in our copending provisional patent application with the Ser. No. 60/493,447, PCT/US01/07527 filed on Mar. 8, 2001, and PCT/US02/07199, filed Mar. 8, 2002, each of which is incorporated by reference herein. Of course, it is also contemplated that all cytokinins contemplated herein may be employed as a therapeutic agent for treatment of diseases associated with dysregulation/dysfunction of AMPK, and/or diseases associated with dysregulation/dysfunction of Akt, and contemplated diseases include diabetes, and especially non-insulin dependent diabetes mellitus.

Therefore, in one aspect of the inventive subject matter, a fermentation medium comprises a cytokinin-containing preparation that includes a cytokinin at a concentration effective to increase fermentation of a microorganism, wherein the cytokinin-containing preparation preferably comprises a synthetic cytokinin having a purine heterocyclic base or a pyrimidine heterocyclic base. Suitable heterocyclic bases include various N⁶-substituted adenine or optionally N⁶-substituted guanine bases, and also various (preferably N-) substituted cytidines (e.g., acylated, acetylated), and it is further especially preferred that such cytokinins are present in the fermentation medium at a concentration of at least 1.0 microM. Alternatively, the cytokinin-containing preparation may also comprise a plant extract (preferably from a plant of the genus Hordeum), or a yeast extract (preferably from Saccharomyces spec.).

While not wishing to be bound by any theory of mechanism, it is generally contemplated that the cytokinin is present in the fermentation medium at a concentration effective to activate an AMP-activated protein kinase of the microorganism, and/or at a concentration effective to increase uptake of a carbohydrate into the microorganism.

Particularly preferred microorganisms include various yeasts, molds, fungi, and bacteria, most preferably of the genera Saccharomyces, Escherichia, and Zymomonas. While not limiting to the inventive subject matter, preferred fermentation media are generally a liquid (and most preferably an aqueous solution or suspension comprising a carbohydrate) or a dough for preparation of a baked good. Therefore, particularly preferred fermentation media include those for ethanol and/or carbon dioxide production (e.g., for fuel generation or beverage production).

Thus, a method of increasing fermentation of a microorganism will include one step in which a cytokinin-containing preparation and a fermentation medium are provided. In another step, the fermentation medium is combined with the cytokinin-containing preparation, wherein the cytokinin-containing preparation is present the fermentation medium in an amount effective to increase fermentation of a microorganism.

Furthermore, it should be recognized that numerous products may be marketed in which it is advertised (typically in printed or otherwise visually perceptible manner) that a cytokinin increases fermentation of a microorganism. Such products typically include cytokinins, cytokinin-containing preparations, brewery kits or components thereof, bakery products, and especially baker's yeast preparations or baking additives, etc. Further contemplated products especially include fermentation media for growth of microorganisms, and ingredients for production of such media.

Thus, specific embodiments and applications of methods and compositions for increasing fermentation of a microorganism have been disclosed. It should be apparent, however, to those skilled in the art that many more modifications besides those already described are possible without departing from the inventive concepts herein. The inventive subject matter, therefore, is not to be restricted except in the spirit of the appended contemplated claims. Moreover, in interpreting both the specification and the contemplated claims, all terms should be interpreted in the broadest possible manner consistent with the context. In particular, the terms “comprises” and “comprising”, should be interpreted as referring to elements, components, or steps in a non-exclusive manner, indicating that the referenced elements, components, or steps may be present, or utilized, or combined with other elements, components, or steps that are not expressly referenced.

We claim:

1. A method of increasing fermentative conversion of a compound that is present in a fermentation medium to a desired product, comprising:
adding a cytokinin-containing preparation to the fermentation medium at a concentration effective to increase the fermentative conversion as compared to the fermentation medium without the cytokinin-containing preparation; and
incubating a microorganism in the fermentation medium to convert the compound.

2. The method of claim 1 wherein the cytokinin-containing preparation comprises a cytokinin at a concentration of at least 1 microM.

3. The method of claim 1 wherein the cytokinin-containing preparation comprises a synthetic cytokinin having a purine heterocyclic base or a pyrimidine heterocyclic base.

4. The method of claim 3 wherein the purine heterocyclic base is an N⁶-substituted adenine or an optionally N⁶-substituted guanine.

5. The method of claim 3 wherein the synthetic cytokinin is selected from the group consisting of N⁶-benzyladenine, N⁶-benzyladenosine, N⁶-benzyladenine-3-glucoside, N⁶-benzyladenine-7-glucoside, N⁶-benzyladenine-9-glucoside, N⁶-benzyl-9-(2-tetrahydro-pyranyl)adenine, N⁶-benzyladenosine-5′-monophosphate, N⁶-gamma, gamma-dimethyl-allyl-aminopurine, dihydrozeatin, dihydrozeatin riboside, dihydrozeatin-7-beta-D-glucoside, dihydrozeatin-9-beta-D-glucoside, dihydrozeatin-O-glucoside, dihydrozeatin-O-glucoside riboside, dihydrozeatin riboside-5′-monophosphate, dihydrozeatin-O-acetyl; N⁶-isopentenyladenine, N⁶-isopentenyladenosine, N⁶-isopentenyladenosine-5′-monophosphate, N⁶-isopentenyladenine-7-glucoside, N⁶-isopentenyladenine-9-glucoside, 2-methylthio-N⁶-isopentenyladenosine, 2-methylthio-N⁶-isopentenyladenine, 2-thio-N⁶-isopentenyladenine, 2-benzylthio-N⁶-isopentenyladenine, 2-isopentenylamine, kinetin, kinetin riboside, kinetin-9-glucoside, kinetin riboside-5′-monophosphate, meta-topolin, meta-topolin riboside, meta-topolin-9-glucoside, ortho-topolin, ortho-topolin riboside, ortho-topolin-9-glucoside, trans-zeatin, trans-zeatin riboside, cis-zeatin, cis-zeatin riboside, trans-zeatin-7-glucoside, trans-zeatin-9-glucoside, trans-zeatin-O-glucoside, trans-zeatin-O-glucoside riboside, trans-zeatin riboside-5′-monophosphate, trans-zeatin-O-acetyl, 2-chloro-trans-zeatin, 2-methylthio-trans-zeatin, and 2-methylthio-trans-zeatin riboside.

6. The method of claim 1 wherein the cytokinin is present in the fermentation medium at a concentration of at least 5.0 microM.

7. The method of claim 1 wherein the cytokinin-containing preparation comprises a plant extract.

8. The method of claim 7 wherein the plant is a member of the genus Hordeum.

9. The method of claim 1 wherein the cytokinin is present in the fermentation medium at a concentration effective to increase uptake of a carbohydrate into the microorganism.

10. The method of claim 1 wherein the microorganism is a yeast and a member of the genus Saccharomyces.

11. The method of claim 1 wherein the fermentation medium is a liquid and wherein the desired product is ethanol or carbon dioxide.

12. The method of claim 11 wherein the liquid is a beverage for human consumption.

13. The method of claim 1 wherein the fermentation medium is a dough and wherein the fermentation comprises production of carbon dioxide.

14. The method of claim 1 wherein the compound is a carbohydrate.
